(19) United States
(12) Patent Application Publication
(10) Pub. No.: US 2010/0005853 A1
(43) Pub. Date: Jan. 14, 2010

(54) Continuous Range Hydrogen Sensor

(75) Inventors: Thomas Visel, Austin, TX (US); Prabhu Soundarrajan, Valencia, CA (US); Igor Pavlovsky, Cedar Park, TX (US); Mohshi Yang, Austin, TX (US)

Correspondence Address: Matheson/ Keys PLLC, 7004 Bee Cave Rd., Austin, TX, 78746, US

(73) Assignee: NANO-PROPRIETARY, INC., Austin, TX (US)

(21) Appl. No.: 11/995,263

(22) PCT Filed: Aug. 3, 2006
(86) PCT No.: PCT/US06/30314

§ 371 Date: Sep. 15, 2009

Related U.S. Application Data
(60) Provisional application No. 60/705,294, filed on Aug. 3, 2005.
(60) Provisional application No. 60/728,353, filed on Oct. 19, 2005.
(60) Provisional application No. 60/728,980, filed on Oct. 21, 2005.

(51) Int. Cl. G01N 33/20

(52) U.S. Cl. 73/19.07

(57) ABSTRACT

A device for sensing hydrogen based on palladium or palladium alloy nanoparticles, wherein the nanoparticles are deposited on a resistive substrate, to permit sensing of less than 1% hydrogen; wherein the nanoparticles are deposited as islands on a continuous resistive layer.

This application claims priority to the following provisional patent applications: 60/705,294 filed on 3 Aug. 2005; 60/728,353 filed on 19 Oct. 2005; and 60/728,980 filed on 21 Oct. 2005.

### TECHNICAL FIELD

The present invention relates to nanoparticle alloy hydrogen sensors.

### BACKGROUND INFORMATION

Palladium is a metal with a property that it readily absorbs hydrogen into its lattice, generally with the result that the lattice expands in size. That expansion is on the order of a few percent at maximum. Various methods have been proposed to take advantage of that fact to sense hydrogen.

A palladium lattice will not grow in length by 5% merely because it is exposed to hydrogen. Rather the expansion occurs only to the degree that hydrogen diffuses into the palladium. Because it cannot diffuse much below the surface, materials are used whose thickness is on the order of that surface penetration depth.

There have been two means of substance devised to take advantage of that “lattice expansion” of palladium. The first is a thin conductive film of palladium, whose electrical resistance increases with increasing doses of hydrogen. A second method is a “nano-wire” technique.

### Thin Film Sensor

Sensors made by this technique place a thin film of palladium between two electrical contacts. Upon exposure to hydrogen, the electrical resistance between the contacts increases. This technique is deemed unstable and difficult to realize in a commercial product (for sensing hydrogen <5000 ppm). Its characteristics are largely defined by FIG. 5 shown later. The available signal change is quite small.

### Nanowire Hydrogen Detector

A tiny wire is created of loosely connected nano-particles of palladium, and placed between two electrical contacts over an insulating substrate. When these expand in the presence of hydrogen, they create electrical shorts between them, effectively closing a switch between the contacts. This is not a sensor, but a “detector” of hydrogen. That is, it does not measure the quantity of hydrogen, but simply its presence.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 illustrates a graph of aging in air and in oil;

FIG. 2 illustrates an exemplary two dimensional calibration chart;

FIG. 3 illustrates a graph of permeability of metals to hydrogen;

FIG. 4 illustrates a graph of an impact of alloy composition in hydrogen in oil;

FIG. 5 illustrates phase change curves for Pd—Ag alloys of different compositions;

FIG. 6 illustrates solubility of hydrogen in metals;

FIG. 7 illustrates phase change curves for Pd—Ag alloys at different temperatures;

FIG. 8 illustrates phase change curves for pure Pd at different temperatures;

FIG. 9 illustrates response-time dependence on operational temperature;

FIG. 10A illustrates a sensor element;

FIG. 10B illustrates a sensor pair with a titanium reference element;

FIG. 10C illustrates a sensor pair, wire-bonded to a carrier PC board;

FIG. 10D illustrates a solid-pattern active element;

FIG. 10E illustrates a striped-pattern active element;

FIG. 11 illustrates a graph of a response of smaller size, lower density 100 percent PdH₂ sensor;

FIG. 12 illustrates calibration curves for a 100% PdH₂ sensor of smaller size, normal density;

FIG. 13 illustrates a response of a smaller size, higher density 100% PdH₂ sensor;

FIG. 14 illustrates a response of normal size, normal density 100% PdH₂ sensor;

FIG. 15 illustrates a SEM micrograph showing variation in particle size and density;

FIG. 16 illustrates a SEM micrographs showing varying the size of sensor elements;

FIG. 17 illustrates a response of a typical sensor with 70-100 nanometer particle size in oil to varying concentrations of hydrogen and temperature;

FIG. 18 illustrates a response of a typical sensor 70-100 nanometer particle size in air to varying concentrations of hydrogen;

FIG. 19 illustrates a two-step plating process and change in conductivity;

FIG. 20 illustrates a safe-operating area curve; and

FIG. 21 illustrates a typical calibration measurement cycle.

### DETAILED DESCRIPTION

In the following description, numerous specific details are set forth such as specific word or byte lengths, etc. to provide a thorough understanding of the present invention. However, it will be obvious to those skilled in the art that the present invention may be practiced without such specific details. In other instances, well-known circuits have been shown in block diagram form in order not to obscure the present invention in unnecessary detail. For the most part, details concerning timing considerations and the like have been omitted inasmuch as such details are not necessary to obtain a complete understanding of the present invention and are within the skills of persons of ordinary skill in the relevant art.

Refer now to the drawings wherein depicted elements are not necessarily shown to scale and wherein like or similar elements are designated by the same reference numeral through the several views.

As an embodiment of the present invention, rather than a linear nano-wire approach, an a real coating of random Pd nanoparticles is fabricated on a resistive substrate. As the particles expand, they short out miniscule resistances in the substrate, which happen to lie beneath two adjacent nanoparticles. On a large-scale statistical basis, the end-to-end resistance of the substrate then decreases in proportion to the amount of hydrogen. This sensor therefore measures a quantity of hydrogen, rather than just detecting its presence. The output signal may be fairly large, often a 2:1 change in resistance or more.

This method yields a greater and more stable signal than either of the thin film sensor or the nanowire hydrogen detector previously described. The nanowire sensor is difficult to fabricate, non-functional at high temperatures, and its “trip point” is difficult to repeatedly establish. By contrast, the sensors of the present invention, whose fabrication techniques are described herein, are repeatable in a commercial production environment. Rather than using vacuum deposition to create a thin continuous film, as for the above thin film sensor, nanoparticles of palladium are created by a controlled electro-plating process. Both the density and the size of the particles can be controlled to yield a repeatable process. This system is also manufacturable in large quantities utilizing current state of the art manufacturing techniques, unlike the nanowire approach.

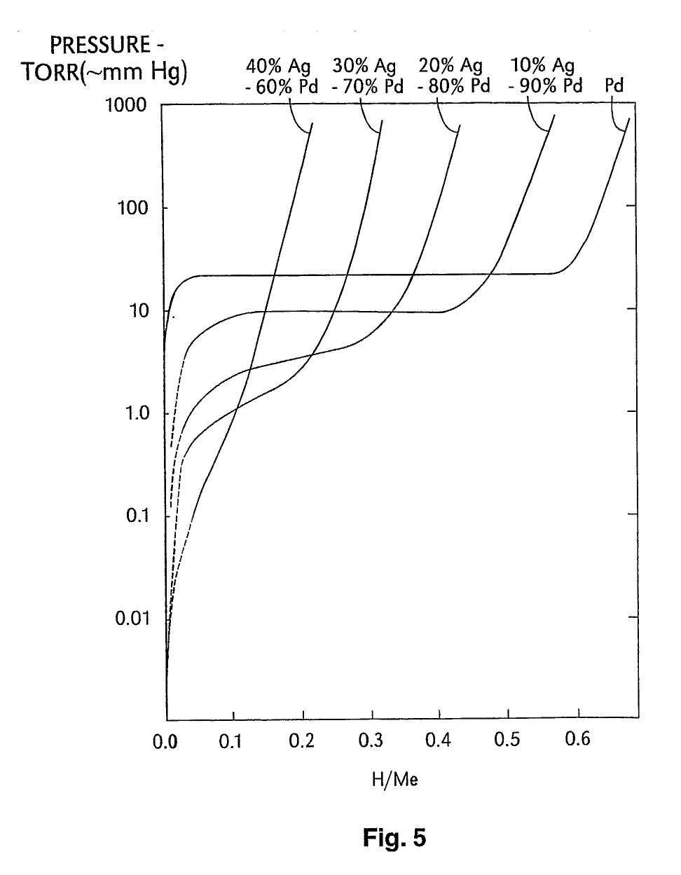

The resistance of palladium films is directly controlled by the characteristics shown in FIG. 5. In an embodiment of the present invention, it is controlled primarily by an underlying resistive substrate. The result is more predictable and stable. In the thin film sensor, quiescent resistance is controlled by uniformity of an expensive film. In an embodiment of the present invention, it is controlled by a low-cost well understood film to which low-cost Pd nanoparticles are attached.

In comparison to other plating and deposition methods, an embodiment of the present invention uses a 20-micron gap from all metal edges to an actively plated area of the sensor. This has an effect during plating of suppressing effects from metal-edge E fields, and yielding a vastly more uniform distribution of particle size, density and a repeatable sensor.

Surface uniformity and cleanliness is important in the sensor fabrication. Application of a washable organic overcoat (X-film) to protect the sensor greatly improves fabrication yields.

As indicated later, long-term stability of palladium nanoparticle sensors is altered by the palladium-silver (Pd:Ag) alloy ratios. Use of alloys according to FIG. 5 is normally a consideration for the linearity of the resulting sensor over ranges of H₂ concentrations. In an embodiment of the present invention, it is used to control the stability of the sensor under stressful environments.

For applications in which slower response times are acceptable (e.g., measurement of hydrogen within transformer coolant oil), this can be used to advantage. For example, sensors without heavy silver content are subject to permanent hydrogen capture (palladium hydriding) when exposed to large temperature drops in the presence of hydrogen. Those with such alloying are more robust against such capture.

Further, the simultaneous exposure to high temperature and high concentrations of hydrogen (e.g., 2% and above) can deform adjacent nanoparticles due to “squeezing” stress. After such exposure, they do not return to their original form, or do so only very slowly. Use of high alloying makes the sensor more robust under such conditions, preventing the altering of their characteristics.

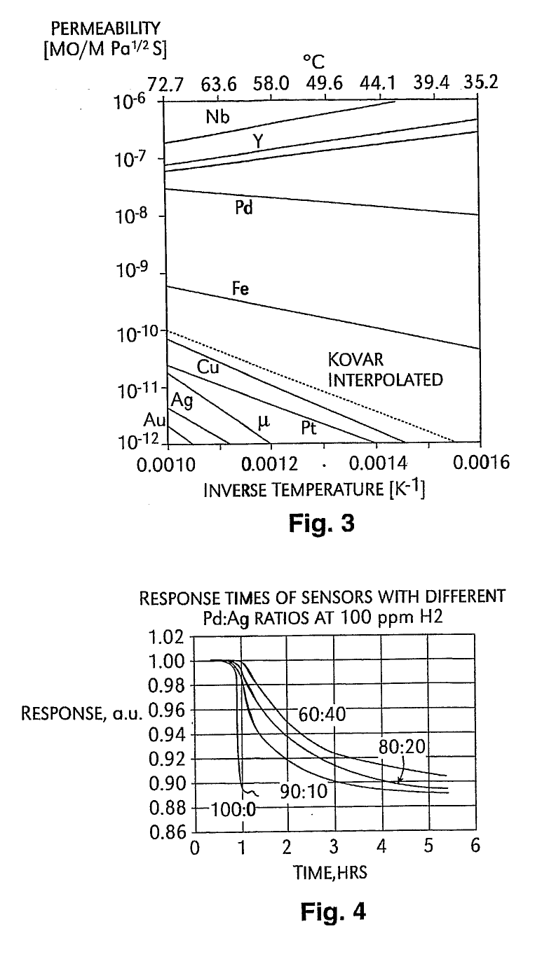

While many physical parameters of palladium (such as shown in FIGS. 3 and 5) are well understood, their application for the creation of a dual purpose commercial hydrogen sensor sensitive at concentration levels less than 5000 ppm in air and oil is not obvious to one skilled in the art.

This disclosure demonstrates how one can create a sensor that is very fast, but of limited dynamic range, or slow and of wide dynamic range. Such alternatives may be selected by varying the Pd:Ag alloy ratios and the inter-particle spacing for the palladium.

It is not obvious to one skilled in the art the impact of the design on its application as an air-based or an oil-based sensor. Nor is it obvious the impact of the rate of temperature changes, and whether they are positive or negative, on the successful characterization of a sensor in oil. This demonstrates such differences and why it is destructive of the sensor to rapidly reduce its temperature while it is charged with hydrogen, such as during characterization procedures.

An embodiment of the sensor of this invention is logarithmic in its response, and can therefore be made sensitive to hydrogen down to a few parts-per-million (ppm). Inasmuch as it is also sensitive to temperature changes, a system of characterization and run-time calibration has been devised and described here, known as 2D calibrations. The system uses interpolation of a set of curves to accurately (within 20% of readings or better) compute the actual concentration of hydrogen present in ppm.

That it is more beneficial to methodically characterize the sensor by stepping the concentration and then cycling the temperatures, versus stepping the temperatures and then cycling the concentration, is not necessarily obvious to one skilled in the art. It provides a two-fold improvement in that certain gains can be had in the simplicity of the test chamber, and lends itself to automated (PID) control.

There are several factors affecting the yield of the disclosed hydrogen sensor.

Factor 1: The underlying titanium metal used to grow the nanoparticles is very reactive to air. The characteristics of the wafer change if the titanium is left exposed to ambient air resulting in variations in the electroplating plating and hence decreasing the yield.

Mitigation: The wafers may be stored in a dessicator with a nitrogen flow to prevent titanium oxidation. An organic coating on the wafer may also provide a mechanism to increase the yield.

Factor 2: The process of scribing individual sticks from the wafer (e.g., 32 sticks/wafer), post-scribing storage, and handling affects the final yield of the sensors since any scratches through the stick affect the electroplating process.

Mitigation: Laser scribing (e.g., CO₂ laser) has been explored extensively with very little success. Excimer laser is an option but is expensive. A simple diamond saw cutter may be efficiently used to maximize the yield of sticks from a wafer. An organic coating over the wafer may prevent scratches on the surfaces of the sticks. A Gelpak may be used for post-scribing storage to prevent scratches due to transport.

Factor 3: The surface cleanliness is an important parameter in determining the efficiency of the nanoparticle electroplating. Surface residues arise generally from the photolithographic process, scribing and handling. In most cases, the sizes of the nanoparticles are smaller than the residue, which are present in manufacturing environments.

Mitigation: The particles from scribing may be removed by a high-pressure air gun; further, a descum process may be employed to help clean the residue from the photolithographic processing.

Factor 4: U.S. Published Patent Application 2004/0238367 describes employing colloidal silver paste as the electrical contacts. This process is crude and non-repeatable. The colloidal silver paste also disintegrates above 70° C. and changes the sensor characteristics. Furthermore, there are no active contact pads in the sensor design. This decreases the yield and performance of the sensors in air and oil.

Mitigation: Active contact pads may be used to electrically connect the nanoparticles. The contact pads may be wire bonded to the sensor holder and the wire bond protected using a temperature stable epoxy. The performance and yield considerably increase as a result.

### Use of Palladium Nanoparticles Versus Thin Films or Nanowires

A thin film of palladium is a continuous surface, with normal metallic connections between atoms. The response of thin-film palladium to increasing levels of hydrogen has a positive coefficient. That is, the resistance increases with increasing concentrations, and directly follows the non-linearities and curves of FIG. 5.

The resistance of a palladium nanowire decreases with increasing exposure to hydrogen, and similar to a low-resistance switch. The switch is closed when the nanoparticles expand and touch each other along the entire length of the wire. It is relatively insensitive to gradations in concentration. The resistive response of the palladium nanoparticles of embodiments of the present invention is a gradual decrease in resistance upon increasing exposure to hydrogen. Unlike the tuning of linearities such as in FIG. 5 through use of alloys, such alloys have a secondary effect upon sensor linearity.

### Use of a Resistive Substrate and Palladium “Nano-Switches”

The present invention places nanoparticles on a resistive substrate, such that the nanoparticles do not touch each other, for the most part. Upon exposure to hydrogen, the particles expand in size (up to approximately 5% of their diameter) and begin to touch each other. Upon touching each other, they short out the region of resistance on the substrate to which they are attached, incrementally reducing the overall end-to-end resistance of the substrate.

Because the particles form a random network and are of random size, the shorting does not occur at a specific concentration of hydrogen, as for the case of nanowires. Rather, the overall resistance gradually decreases as the exposed hydrogen concentration increases.

### Characteristics of a Resistive Layer

Certain requirements are imposed on the resistive layer on which the nanoparticles may be formed. It should be stable with temperature, should be insensitive to environmental factors, and should accept the formation of the nanoparticles.

It further yields a certain “non-exposed” resistance that is optimal for the electronics to which it connects. For the case of the sensors and electronics of embodiments of this invention, a resistance of a 0.5 mm×2.0 mm resistive surface yields a resistance of 1200 to 2200 ohms.

An optimum value is determined by desired operating current, impedance-based immunity to nearby electrical signals, and by resistive stability of the surface. If a surface such as titanium is used, thicker surface films improve aging characteristics but diminish both resistance and available signal. If that same film is too thin, electrical noise increases, and the film is less immune to effects such as oxidation, for which titanium is otherwise notorious.

An exemplary resistance for the above physical configuration is 90 to 150 angstroms (titanium).

Another optional film, for example is vanadium. Because it has a lower a real (sheet) resistance, the vanadium film thickness is less than that for titanium. It has an advantage over Ti in that it is less subject to oxidation. It may be somewhat more difficult to work with than titanium.

The actual choice of resistive film material does not alter the means and methods of embodiments of this patent. Each material brings with it physical characteristics that can be compensated for using the general means of embodiments of this patent.

### Control of Oxidation and Aging

Time, temperature, exposure to hydrogen and other factors can degrade or otherwise alter the characteristics, such as resistance, of the underlying resistive film. For a titanium film, a primary cause of change (aging) is that of oxidation of the surface.

For a given initial thickness of titanium, the overall thickness changes very little with time. Rather, the pure Ti is gradually replaced at the surface by an equivalent thickness of TiO₂. One means employed in embodiments of the present invention is to pre-expose the Ti surface (after application of nanoparticles) to oxygen at elevated temperatures. This is a “conditioning” step and is taken after device fabrication but prior to calibration of the sensor.

By intentionally thickening the Ti film beyond its optimum value, such a conditioning exposure of Ti to oxygen replaces part of that thickness with insulating TiO₂, which itself is relatively stable. As the latter insulating layer thickens, diffusion of oxygen to the lower layer is gradually impeded. The result is an asymptotic approach of the element's resistance to some stable value.

It should be noted that oxidation of the Ti film takes place after palladium nanoparticles are grown on it, and does not appear to significantly undermine the Pd—Ti boundary. Further, it does not appear to lessen the adhesion of the palladium to the Ti film.

An alternative approach is to use a material such as vanadium that has a higher stability in air compared to titanium.

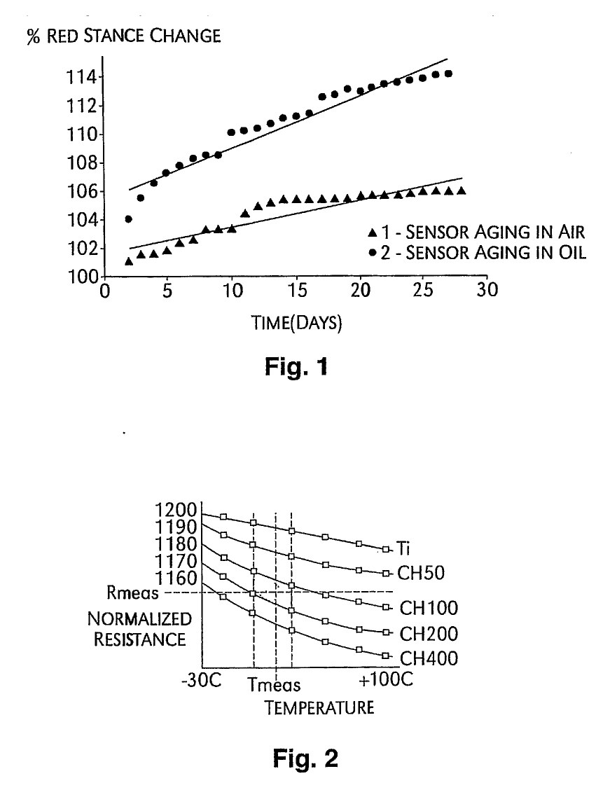

FIG. 1 shows an early curve of unmitigated aging in a sensor under two conditions of storage in air and in oil. The asymptotic effects of oxidation can be seen. The non-uniformity of the curves is due to the manual methods used in the measurements, and is smooth when automated measurements are taken.

### Use and Choice of a Reference Element

For resistive layers subject to oxidation, such as for titanium, something may be done to prevent or account for resistive changes with aging, or to compensate for them. Embodiments of the present invention may use several techniques to do this.

First, a “reference” resistive element may be created alongside the active palladium-coated element, and used to compensate for changes in resistance. It was not obvious to the inventors how to create two electrically similar elements, yet have only one of them sensitive to hydrogen. Few materials are known that can block diffusion of hydrogen into the reference sensor. Almost every blocking method, technique or material has its own serious drawbacks.

In an embodiment of the present invention, a method used is to simply not apply nanoparticles to the reference element. While resistive changes due to temperature differ slightly between the two elements, these can generally be compensated for. The reference element that is free of palladium simply does not respond to hydrogen.

### Temperature Dependence and its Correction

Three primary factors give temperature dependence to the sensor. These are
Change in substrate resistance (minimal, in Ti)
Change of particle diameter with temperature
Change of substrate surface area with temperature

Of these, the second two are important. Increases in temperature expand the palladium particle diameter, potentially causing adjacent particles to short. This decreases the effective resistance of the sensor, identically to a hydrogen response.

The substrate surface area may also increase with temperature. If the linear growth in any dimension exactly matches particle diameter growth, there would be no net resistance change. To the degree that surface growth and particle diameter growth are not matched with temperature, the net resistance of the sensor will change with temperature.

A solution is to select a substrate whose Temperature Coefficient of Expansion (TCE) is matched to that of palladium. It may also be matched with the TCEs of the resistive layer and any adhesion layers used.

### Trade-Offs Between Substrates

Various conflicting factors exist for substrate selection. A practical one is the amount of handling required, which directly reflects on sensor yield. For example, sensors can be fabricated on silicon wafers, aligned along the strain lines, permitting ready cleavage and dicing. In one embodiment, sensors are grouped into 5-sensor “sticks.” The wafer is first scribed and fractured into these sticks for palladium processing. After such processing, the sticks are then scribed and diced into individual sensors.

Glass substrates have no orthogonal strain patterns as silicon does, and thus should be scored and fractured carefully, but require use of more force and create more particulate “trash” to clutter the sensor. Proper scribing of glass requires significantly more effort and care.

Any other substrate that can withstand the temperature extremes of the live sensor environment and meets TCE criteria may be alternatively used without altering the teachings of this invention.

### Proper Choice of Substrate and Thermal Matching

For reasons given previously, the thermal matching of the substrate and palladium may be also important. The TCEs of silicon and palladium are not well matched; with the result that sensor resistance changes with both temperature and H₂, rather than with H₂ alone.

In spite of the more critical handling factors, borosilicate glass may be used, being a relatively good thermal match for palladium, titanium and gold. A protective organic overcoat may be applied to sensors prior to final dicing to minimize the impact of scribe trash on the sensor.

### Morphological Stress to the Nanoparticle Sensor

Sensors created under this invention may be considered damaged or altered if their behavior or response to hydrogen or temperature changes after fabrication. Such damage may be minimized by proper surface design, proper design of the nanoparticles, and by proper handling during the conditioning and test phases of production.

In short, any condition that permanently alters the physical geometry of the nanoparticles, or that alters the characteristics of the resistive surface may cause such destruction. Sensors destined for use in oil differ somewhat in these matters than those destined for use in a gas environment.

Two possible means of stressing palladium nanoparticle sensors may be the simultaneous applications of heat and high concentrations of hydrogen, and the rapid reduction of temperature during (or shortly after) exposure to high concentrations of hydrogen.

Consider that adjacent nanoparticles expand under the application of either temperature or hydrogen. If they simply “kiss” and touch each other, they retreat to their original physical shape upon removal of temperature or hydrogen. Sensors must be created with sufficient nanoparticle spacing to allow these changes.

Improper spacing or particle size could cause particles to contact each other under “quiescent” (no hydrogen, room temperature) conditions. Any substantial increase in their size could then cause them to deform, such that they never revert to their quiescent-condition sizes and shapes. In this case, the effective resistance of the particle network is permanently altered. With proper alloy ratios, even this deformation can be mitigated to a significant degree, especially with higher Pd:Ag ratios such as 60:40.

Should a sensor be so altered or damaged, it may be possible to recalibrate it and restore it to usefulness. That is, it is still sensitive to hydrogen, but is now nonlinear with respect to its original resistance versus H₂ and temperature calibrations.

### Morphological Stress of the Nanoparticle Sensor

A second form of damage to the sensor of this invention involves possibly permanent change of sensitivity due to chemical change, and relates to diffusion rates. It results in the (probably irreversible) formation of palladium hydride.

When hydrogen has diffused into the palladium nanoparticles in a gaseous environment, rapid reduction of temperature causes a shrinking of the particle. This is simply the normal return to size at (for example) room temperature conditions.

As the particles shrink, pressure on the absorbed hydrogen within is implied, whether via charge opposition or other mechanisms. Hydrogen will therefore diffuse out (“exfuse”) of the particles. Given a simultaneous reduction in hydrogen concentration in the environment, hydrogen will diffuse out even more rapidly. These are normal behaviors.

In other circumstances, and upon rapid drop in temperature, exfusion could be inhibited or severely restricted. This could happen, for example, if the surrounding environment is very dense, such as when measuring hydrogen dissolved in oil. The oil molecules may block the normal exfusion of hydrogen, trapping it within the palladium for a time.

When this happens, the internal pressures on the infused hydrogen would be substantial. It would appear under these conditions that a chemical reaction is encouraged, resulting in the probable (non-reversible) formation of palladium hydride. The result is that the particles never return to their quiescent-condition sizes, causing a permanent change in resistance.

Solutions to this issue are to never permit such a rapid temperature change while the sensor is hydrogen-charged, or to alter the morphology or substrate TCE matching.

### Calibration of a Sensor

The sensors of embodiments of the present invention respond in some manner to both hydrogen and temperature. It is not sufficient to measure the resistance and compute the equivalent level of hydrogen. Rather, a series of curves are created for the calibration of a sensor, curves which may be specific to each sensor. For selected levels of hydrogen within the range of the sensor, measurements are made across a range of temperatures.

FIG. 2 depicts an example 2D calibration chart. It shows data taken at four H₂ concentrations, with measurements at each concentration being made over the −30° C. to +100° C. temperature span.

A temperature sensor is co-located with the H₂ sensor and selects a vertical line on the above chart. Measurement is made of the present sensor resistance itself Interpolation is then used to determine actual H₂ content.

### Proper Palladium-Silver Alloy Ratios

For various reasons, the nanoparticles grown on the sensor surface may not be pure palladium, but may be an alloy of another metal such as silver or nickel. Choice of the alloy ratio impacts speed of operation and robustness of the particles.

A heavier alloy (e.g., a Pd—Ag ratio of 60:40), for example, creates a sensor that is relatively immune to morphological stress-induced changes. This permits use in oil under a wide range of temperatures and H₂ concentrations. A downside of this ratio is that it has a much slower response. In a transformer-oil environment and application, though, this is normally not an important factor.

A lighter alloy yields a sensor that is faster, although it is not as robust in an oil environment. It is more suitable for applications in a gaseous environment, such as in fuel cells and in open air. In these gaseous environments and applications, however, morphological stress-induced changes are not an important factor.

### Hydrogen Permeability of Metals

In the case of alloying Pd with Ag in nanoparticles, the sensor response time depends on the hydrogen diffusion process through both palladium and silver, i.e., on permeability of these metals to hydrogen. The literature data shown in FIG. 3 illustrate permeabilities (at elevated temperatures) of Pd, Ag and other metals to H₂.

FIG. 3 shows that the permeability of Ag is at least four orders of magnitude less than that of Pd. Therefore, even small amounts of Ag can significantly change the response time of a sensor that uses a Pd:Ag alloy. The experimental evidence of this has been observed with Pd:Ag nanoparticle sensors as well. The data below have been obtained for the sensors operated in transformer oil.

The FIG. 4 chart shows response times for pure Pd and alloyed nanoparticles sensors: Pd:Ag ratios of 100:0, 90:10, 80:20 and 60:40. The response of a pure Pd nanoparticle sensor to hydrogenated oil is some 6-10 minutes, but take some 4 hrs for a 90:10 sensor, some 5 hours for an 80:20 sensor, and over 6 hrs (with no stabilization seen on this plot) for a 60:40 alloy and 100 ppm H₂.

Response Time versus Concentration

The sensor response time depends upon both alloy composition and the hydrogen concentration. For a concentration of 1000 ppm in oil, the response time will be ˜9 minutes for a 90:10 alloy, and ˜15 minutes for a 60:40 alloy.

Summarizing, an embodiment for faster response hydrogen sensor involves Pd:Ag ratios of 90:10 to 99:1 and lower Ag content. These find usage in gaseous environments. Slower response sensors involve Pd:Ag ratios of higher Ag part than 90:10, preferably 80:20, and even more preferably Pd:Ag=60:40. These find usage in oil-based environments.

### Nanoparticle Plating Conditions

Electroplating the nanoparticles is beneficial. Successful operation of the sensor is enabled when the nanoparticles have a certain distance between each other within a narrow distance window.

If inter-particle spacing is large, the sensor will be both slow and insensitive to low concentrations. Indeed, there will be a minimum threshold, for both temperature and pressure, below which the sensor will not function. This is because the particles are spaced too far apart to touch each other, even at their times of greatest expansion and growth.

It is therefore important to control both the nano-particle size and the seeding density on the substrate. In an embodiment of the present invention, palladium nanoparticles are grown by a plating process in which both nucleation and growth parameters are controlled. The electroplating may be a constant current (chronopotentiometry) or a constant voltage process (chronoamperometry). The electroplating process may be a two step process involving a short nucleation pulse (<10 sec) and a growth pulse (<10 minutes). The density of the nanoparticles are generally dependent on the charge applied during the nucleation step, and the applied charge during the growth step regulates the size of the nanoparticles.

Sensitivity of the Sensor versus Alloy Ratio

Alloy composition affects not only the response times but also the range of sensitivity of hydrogen sensors. It is further disclosed how addition of silver improves the sensitivity of sensors at low hydrogen concentrations.

Given conventional phase-change curves, such as in FIG. 5, it could appear that sensitivity depends on alloy composition. However, a principle of the sensor operation shows that the sensitivity depends more strongly on other factors instead.

To consider the sensitivity as a response of sensor to a given concentration of hydrogen, consider the dimensional response of a single nanoparticle to hydrogen, taking into account the collective effect into the sensitivity of the nanoparticle network.

Considering an individual nanoparticle, an increase in the nanoparticle size (i.e., phase change) is proportional to the amount of Pd material in the nanoparticle. That is, nanoparticles with higher Ag content change size to a lesser degree than pure Pd nanoparticles. This dependence is not linear, as illustrated graphically in FIG. 5.

From FIG. 5, it is readily seen that the dependence of hydrogen accumulation in a nanoparticle is a complicated function of alloy composition. The dependence is almost linear with the Pd content at higher hydrogen pressures, but this reverses as the partial H₂ pressure drops below ˜20 torr (˜2.5% in air).

Further, the dependence on H₂ with higher Ag content becomes smoother than for pure Pd and even a 90:10 alloy. This enables the creation of continuous range hydrogen sensors.

The sensors are more sensitive (the alloy absorbing more hydrogen) at lower H₂ concentrations if they have more silver in the alloy, as can be seen from the “40% Ag-60% Pd” curve in FIG. 5.

The hydrogen absorbed in this alloy at ˜0.01 Torr is approximately the same as the amount absorbed by the “10% Ag-90% Pd” alloy at ˜1 Torr. This makes sensors using 40:60 alloys preferable for lower-level hydrogen detection applications.

### The Impact of H₂ Solubility in Metals

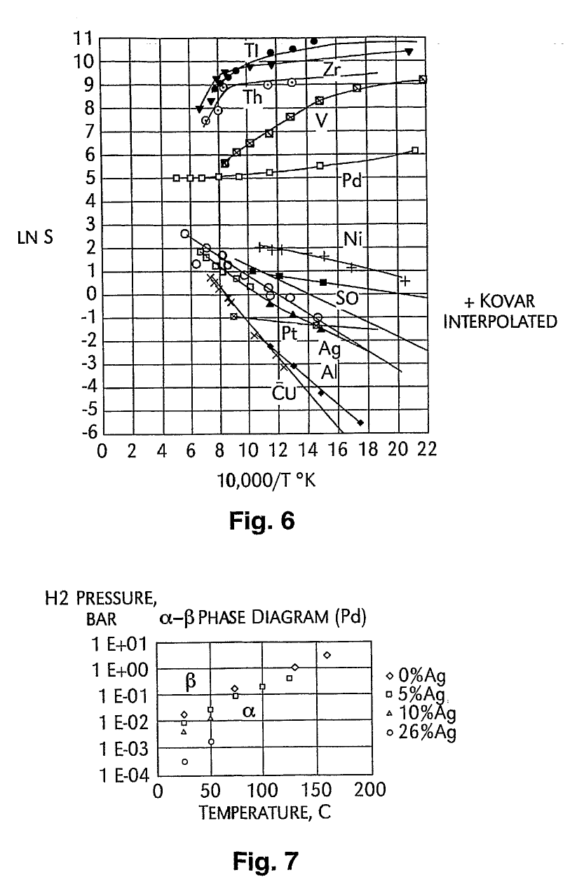

Another factor impacting sensor response to H₂ is the solubility of hydrogen in an alloy. FIG. 6 shows solubility of hydrogen in different metals. Hydrogen is notably more soluble in palladium than in silver, by several orders of magnitude. This difference also affects the integral hydrogen uptake by a nanoparticle.

### Temperature Dependency of Sensor

Just as sensor sensitivity depends on the alloy composition, it depends on the temperature of sensor operation. This is due to dependence on temperature of solubilities of hydrogen in metals.

FIG. 7 shows how the phase change depends on the temperature at different H₂ concentrations for different alloys.

For different alloys, the dependence vs. temperature is similar in trend, and indicates that the detection of lower hydrogen concentrations requires lower operational temperatures for any given alloy. The alloy with higher Ag content may be preferable for detecting low levels of hydrogen to any alloy at any fixed temperature.

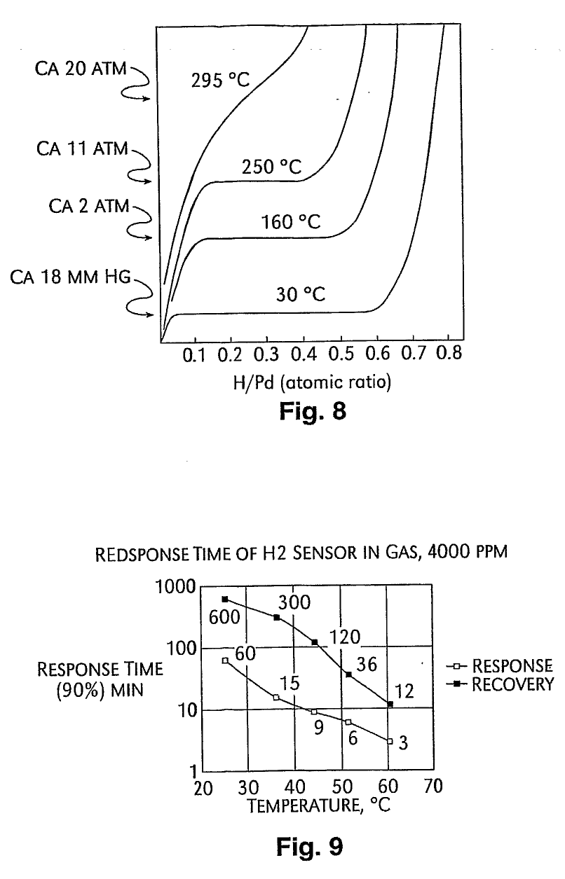

For reference, FIG. 8 shows phase change temperatures for different pressures of H₂ in pure palladium. Notice that unless certain changes in the sensor design are made, the temperature interferes with the sensitivity to hydrogen, since both result in an increase of size of nanoparticles. At higher temperatures and higher H₂ concentrations, the nanoparticles expand.

### Cross-Sensitivity to Hydrogen and Temperature

This interference requires calibration of the sensor against hydrogen at different temperatures, as previously discussed for FIG. 2. Therefore, measurements are taken over a range of temperatures to produce a set of calibration points. This approach ensures better accuracy of the sensors. An alternative method, which avoids adjustment of sensor readings for temperature, keeps the sensor heated at constant temperature during operation.

Diffusion Rates with Temperature, and Response Time

Sensor response time depends on the sensor operation temperature, not only because of particle size and spacing issues, but also because of diffusion rates. Response time depends on the diffusion of hydrogen through metals as well as on the concentration of hydrogen surrounding the nanoparticles. (Note, the permeability itself is a derivative of a diffusion coefficient as shown in FIG. 3.)

The diffusion coefficient typically increases with temperature. For material without defects (to which Pd:Ag nanoparticles are not referred), the diffusion depends on T as

D=D₀ exp(−A/kT),

where D₀ and A are constants.

This indicates that the response time should also decrease as the temperature increases:

t˜1/D˜exp(A/kT).

FIG. 9 shows the measured dependence of the response time of a hydrogen sensor with Pd:Ag composition of 80:20 in hydrogen gas. The flow rate is 260 sccm at 4000 ppm H₂ concentration. In the figure, “Recovery”means the exfusion of H₂ from the sensor as it returns to quiescent conditions.

Over the range of 25 to 60° C., the response time changes by more than an order of magnitude, from 60 min. to 3 min. for a 90% of maximum response, and from 600 min. to 12 min. for 90% of the recovery. The dependence plotted in semi-log scale suggests that the temperature dependence of the response follows the exponential dependence, which is likely a diffusion related phenomenon.

The strong dependence of the sensor response on temperature is useful for making fast-responsive sensors operating at elevated temperatures, where a sensor would incorporate a built-in heater.

As indicated previously, the response time of the sensor is proportional to hydrogen diffusion rates in the sensor, which FIG. 4 shows these to be proportional to the percentage of palladium in the alloy. That is, use of pure palladium yields a faster response to hydrogen than does a high-alloy sensor.

It is well understood that at room temperature the phase transition in pure palladium metal a-phase to b-phase occurs about 1% H₂ at atmospheric pressure. There is no prior art on a hydrogen sensor based on palladium thin films or nanoparticles or nanowires that is operational for <1% H₂ and stable over 60° C. Embodiments of the present invention provide a solution to the problem by a palladium nanoparticle sensor grown on a resistive substrate, where the resistance of the resistive layer changes on the opening and closing of nano-gaps.

The operation of a pure Pd hydrogen sensor at lower parts per million ranges with a temperature range of 0-100° C., presents a considerable improvement over any existent hydrogen sensor based on Pd. The response time of the sensor is faster with smaller particle size and higher density (FIG. 4); the sensor is also more sensitive.

### Plating Nucleation Versus Growth

Previously noted, decreasing the size of the particles to increase response speed requires that the particles be seeded more densely to ensure adjacent particles will contact each other upon expansion. Particle density may be controlled by plating nucleation charge (Current×Time), whereas particle size may be controlled by subsequent growth charge. Nucleation current is much higher than growth current, while nucleation time is much shorter than growth time.

A constant growth current may be applied to establish overall charge. If density is decreased to make particles sparser, but growth time and current remain unchanged, that same current is applied to a smaller number of particles. This means that each particle receives more current, and the particles grown (at a lower density) therefore grow larger. Once an optimum growth charge is obtained for a given particle size, the size/density ratio can be varied over some relatively linear region by altering the nucleation time. That is, the ratio is self-regulating to a large degree, such that there is considerable latitude available in nucleation time (particle size) before compensation of the growth time or current is required.

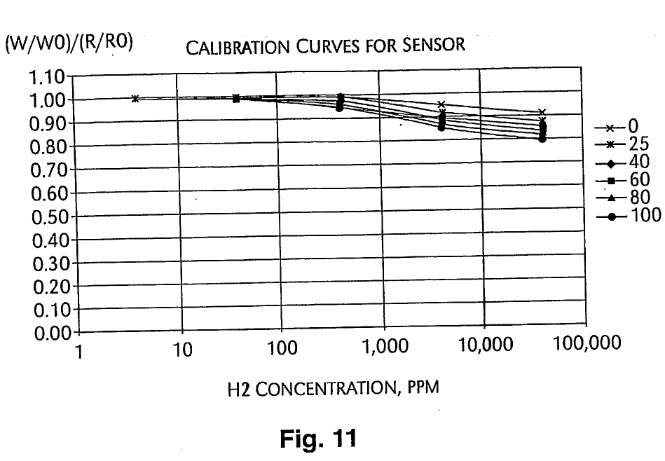

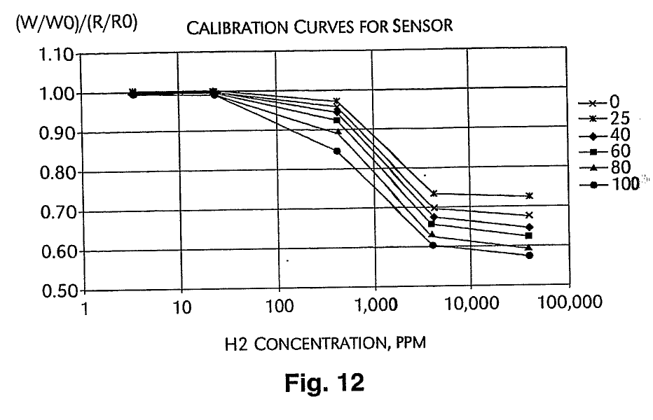

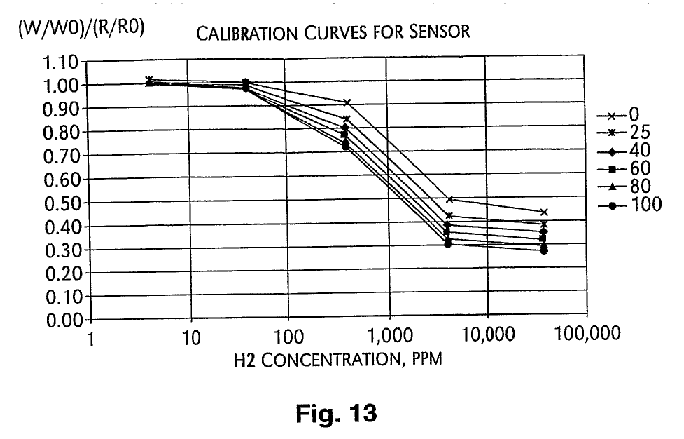

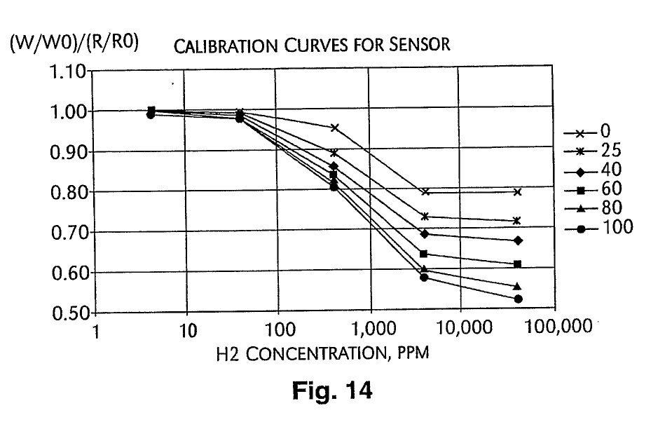

FIGS. 11-14 illustrate effects of various particle sizes and densities.

### Production Process

The following are steps that may be used in production of the sensors of embodiments of the present invention.
Deposit metal layers and Pd mask
   Clean and Prepare the Wafer
   Deposit a titanium or other resistive layer
   Deposit a chromium adhesion layer for gold contacts
   Deposit gold for contacts and plating connections
   Deposit a mask for plating of sensor active areas
Scribe and fracture wafer into sensor “sticks” (optional, for partial-wafer plating)
Fabricate the sensor
   Re-prep of stick or whole-wafer surface
   Plate the sensors (on a stick or whole wafer)
   Pretest sensors in air for operation
   Mount and wire-bond sensors to carrier
Test the Sensors
   Condition the sensors
   Characterize and test the sensors
   Spot-check the calibration

The process details are further described below.

### Sticks or Whole-Wafer Plating

The plating portion of the process can be performed on a regional or whole-wafer basis. The wafer may be partitioned into “sticks” of sensors (and test coupons) each. Plating may be subsequently done on a per-stick basis, for convenience and rapid process development.

It will be obvious to one skilled the art that plating of an entire wafer may be performed, with appropriate controls, rather than a small area at a time as described here. In this discussion, the relevant portions may be so modified, eliminated or deferred, as is appropriate to whole-wafer plating.

### Choices of Substrate Material

Reasons for selection of one substrate material over another were described above. It is convenient to use silicon wafers during the development process, but these are not a good TCE match with palladium. Over the temperature range of choice (e.g., −30° C. to +100° C.), silicon shows the same magnitude of resistance change that 2000 ppm H₂ would show. For this reason, a borosilicate glass substrate (0.55 mm thickness) may be used. One skilled in the art will appreciate that any material of matched TCE that is compatible with the deposition methods and tolerant of the sensor target temperature range may also be used.

### Wafer Preparation

The substrate (glass or silicon) may be prepared to receive metal masks. For the proper and uniform adhesion of nanoparticles on the final sensors, the wafers may be cleaned and treated. The glass and silicon wafers may be cleaned using standard cleaning procedures before the metal deposition steps. The cleanliness of the wafers may be monitored throughout the metal deposition process and protected from impurities/scratching using a commercially available organic coating (X-film). The protective coating may be water stripped before the plating process.

### Deposition of Metal and Pd Mask Layers

The metal and masks used to create the sensors may use conventional photolithographic techniques such as used in the semiconductor industry. Surfaces may be patterned using chrome-on-glass masks and conventional photo-resist processes. While the following descriptions use titanium as the resistive layer, one skilled in the art will appreciate that other materials such as vanadium could also be used. Thicknesses and surface preparations may need to be altered to account for such choice.

After preparation, a uniform (unmasked) film of titanium is laid down with a thickness that will yield sensors in the 1200 to 2200 ohm region. While the thickness may be varied to meet specific resistivity and aging requirements, the Ti layer may be typically 90 to 150 Angstroms. The Ti may be back-etched via a mask to remove material outside the sensor active or connection line/pad areas.

Using a second mask, gold contact pads and external connection traces may be deposited using a mask. Using the same mask, a chromium (Cr) adhesion layer is first deposited, and then the gold. The mask is then washed off to leave Ti and gold-over-chrome.

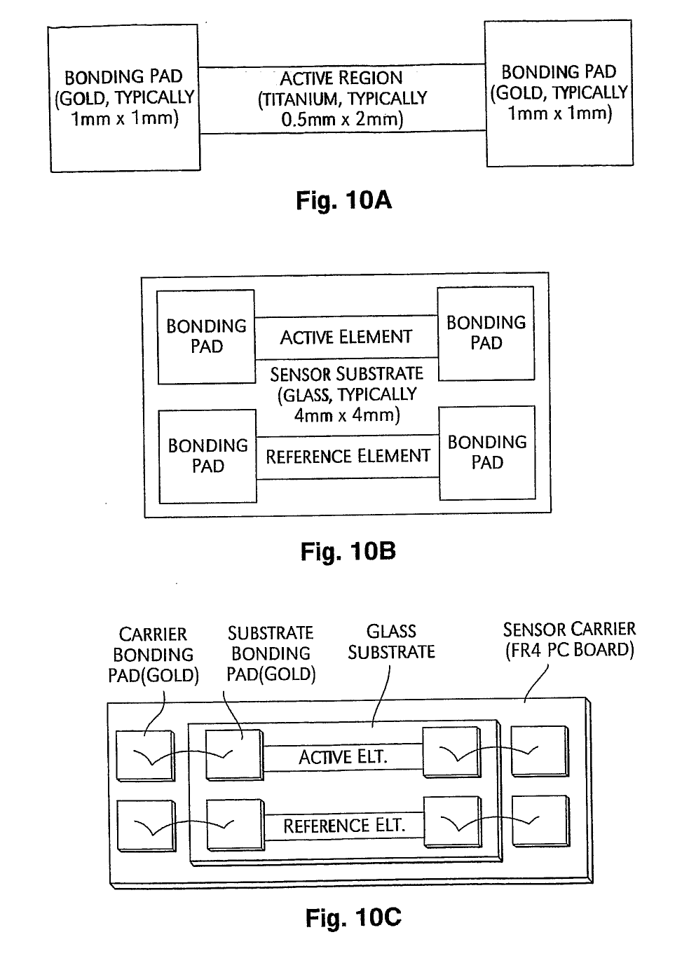

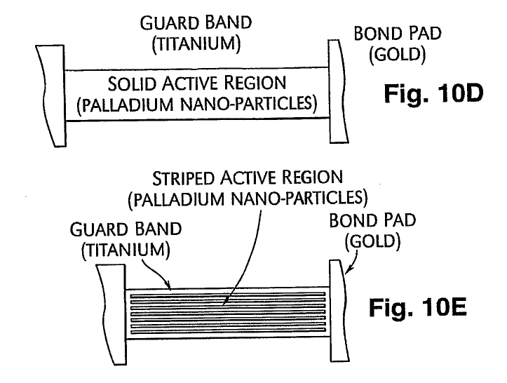

The final step may be to pattern the wafer to leave plating “windows” over the bare Ti that is to be the active portion of the sensor. As described later, this mask leaves a 20-micron Ti gap around the area to be Pd—plated (see FIG. 10D). This gap controls the E-field to inhibit the thickening of Pd plating around the edges.

### Resistive Substrate Deposition Criteria

The choice and thickness of the resistive substrate material is important to the operation of the sensor. A portion of the current sent through that substrate will ultimately be switched on and off by the touching of two adjacent palladium nanoparticles. If that current is excessive, the particles can be destroyed, deformed or in other ways modified by the locally-heavy currents.

Sensor element end-to-end resistance is dependent upon geometric shape and resistive substrate film thickness. For a given resistance, the current is controlled by the voltage applied across the ends.

### Size of Active Area

Referring to FIG. 10A, it has been found convenient to have a 0.5 mm×2 mm (Length/Diameter=4) active area on the sensors. Other sizes have been used, but this is a trade-off between resistance, active area and sensor stability. At each end of this area may be a 1 mm×1 mm gold bonding pad.

The substrate material may be titanium, although this may be replaced with less-reactive vanadium. One skilled in the art will appreciate that various other materials could be used, including organic materials, so long as they fit the resistivity and operational ranges, and material compatibility issues for the sensor as a whole.

### Target Resistance

Current-density calculations show that sensor currents of 20 to 80 μA are good for long-term stable operation. One method of signal sensing is to pass a constant current through the sensor of 20-40 μA, and then read the voltage across the sensor via an A/D converter.

These parameters may be modified to suit the external electronics. Resistances in excess of 2800 Ohms or so may tend to be noisy, implying that the underlying resistive layer is too thin. One range of quiescent resistance is 1200 to 2200 Ohms. This may be achieved using a (Ti) thickness of 90 to 150 Angstroms.

### Oxidation and Aging

Discussed earlier, titanium is a quite reactive metal, and must be well understood to be useful in a sensor application such as this. Referring to FIG. 10B, to compensate for oxidation-based aging of the sensors, a reference resistive element may be added to the sensor. It may be identical to the active sensing element, but may be no palladium plating. Both oxidize at approximately the same rate, and the reference element is used to compensate for residual aging resistance changes.

To minimize oxidation-based aging in the field, the sensors may be pre-oxidized by subjecting them to an elevated temperature in an oxygen atmosphere. For example, the resistive Ti film may be 100 Angstroms thick when created. Oxidation may reduce that thickness to perhaps 80 Angstroms, for example, replacing 20 Angstroms by TiO₂, an insulator.

While the oxidation continues indefinitely, it gradually becomes a much slower process as the oxide thickens, because large O₂ molecules are required to penetrate far deeper than at the start of the process.

To control the aging, the Ti layer may therefore be thickened so that it can be corrected back by the thinning process of pre-oxidizing it. Therefore, thicker films of 150 Angstroms, for example, may be used instead of thinner 90 Angstroms, for example. The trade-off is that it provides a lower initial resistance. FIG. 10C illustrates the sensor pair mounted on a sensor carried PC board.

### Uniformity of Ti Film

Obtaining a uniform resistance across the wafer may require that the Ti film be uniform across the wafer. Use of a thicker Ti film results in greater overall uniformity, but also results in a lower starting resistance. It is important that the Ti resistance effects be much larger than the bulk resistance of the palladium. Again, 120 to 150 Angstroms is a good trade-off for Ti film thickness.

### Shape of Palladium Mask

Referring to FIGS. 10B and 10C, a single sensor may comprise two elements, one active and one for reference. They may be identical in size and shape, except that the reference element is not plated. A 0.5 mm×2 mm resistive area may be used by way of example, but one skilled in the art will realize that other sizes and geometries can be used without altering the means of this invention.

Referring to FIG. 10D, the non-gold (non-pad) region of the active element of the sensor may be covered by a 20 μm mask border to preclude it from being plated. As discussed previously, this prevents E-field effects from causing more aggressive plating near the edges of the element.

### Design of the Reference Element

The reference element (FIG. 10B), may be identical in every way to the active element (FIG. 10B), except that it may not be plated with palladium. The photo mask used to create the palladium plating windows may simply cover the entirety of the reference element during the plating step.

Striped Areas versus Solid Areas

For the active element, two palladium mask types may be used, solid-fill (FIG. 10D) or striped (FIG. 10E). In the solid-fill version, except for the 20 μm borders, the entire active area is plated with palladium. In the “striped” version, various widths of palladium lines may be formed, all over a solid titanium resistive sheet. Nominal line-and-space widths may be 10 μm and 10 μm, respectively.

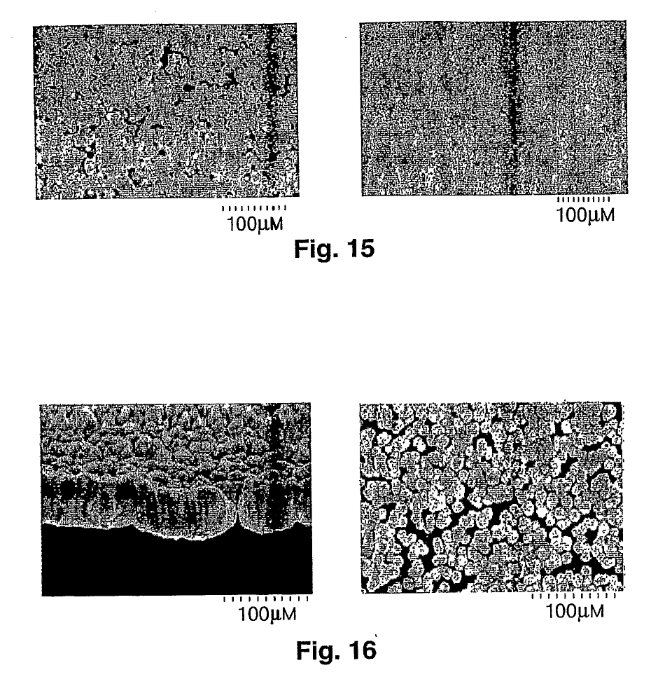

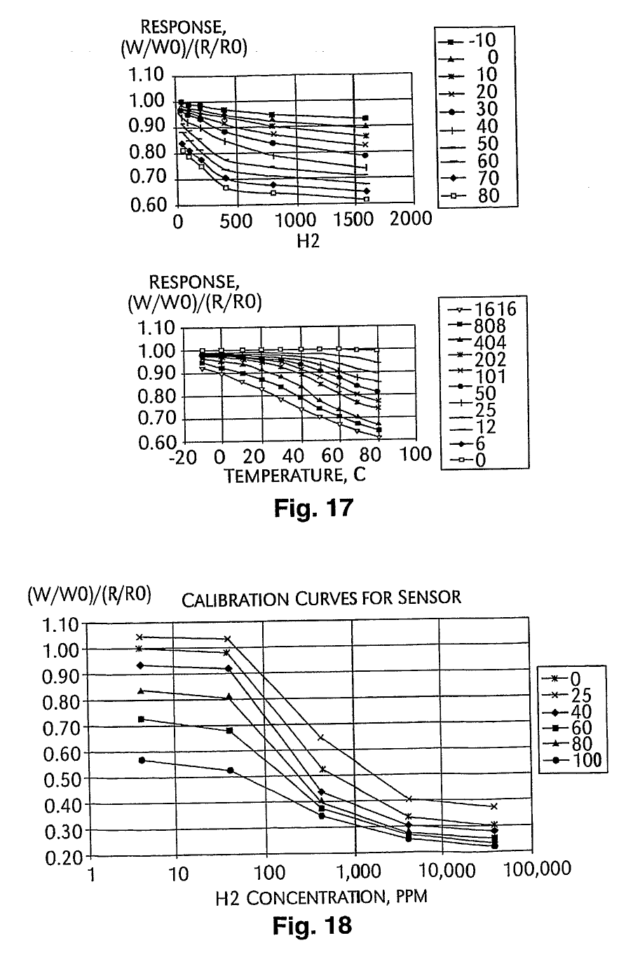

While in both cases, the size of the nanoparticles is similar, the solid versions are consistently less sensitive to hydrogen and are less stable than the striped versions. Using SEM techniques, it has been determined that the nominal size of nanoparticle is on the order of 70-100 min. Another size used is on the order of 35 nm particles, which are plated more densely to compensate for the inter-particle gap. FIG. 15 shows SEM micrographs showing variation in particle size and density. The left micrograph shows 70-100 nm particles, while the right micrograph shows 30 nm particles with much higher density.

Particle Size Variation from Edge into Center vs Resolution Correlation—Stripes vs. Solids

The uniformity and the size of the nanoparticles may affect the resolution of the hydrogen sensor. Different sensor designs have an effect on the edge particle morphology. Different embodiments might be solids (500 μm width lines for nanoparticle), stripes (10 μm wide lines for nanoparticle deposition). Nucleation may be more controllable in the 10 μm wide lines (stripes) in comparison to large 500 μm lines (solids), resulting in more uniform edge to center particle morphology.

Variations from the size of the sensor elements described above have been made. While sensitivity is similar in each case, the effective resistance of the sensor elements change. Below 500 μm, however, plating becomes unstable and more difficult to control, and it is harder to obtain sensor-to-sensor uniformity. The 500 μm element width has been found good for many purposes with the electronics in use.

FIG. 16 SEM micrographs show the variation in particle size and density along the edge. The micrograph in the left shows 500 nm particles along edges (cross-section), and the right micrograph shows 50 nm particles in the center.

### Optimization of Particle Size and Density for Air and Oil Operation

There is an optimum particle size and density for operation in oil in the dynamic concentration and temperature ranges. The concentration levels of interest in oil are in the lower ppm ranges (0-1000 ppm), while the levels of interest in air ranges between 5000 and 50,000 ppm. An optimum particle size for operation in oil without fallback (a phenomenon wherein the sensor response is altered due to excessive plating along the edges resulting in concentrated stress patterns) is around 70-100 nm with a nominal particle density.

Scribing and Fracture into Sticks

If whole-wafer plating is used, this step is not necessary, or is deferred to and incorporated into the sensor dicing step described later.

Especially when glass substrates are used, it may be useful to coat the wafer with an organic material (X-film), to protect it during the scribing and fracture process. This is later washed off and protects the sticks during handling, increasing the yields.

The sensor wafer may be partitioned into 5-sensor sticks at the mask level. Here, it may be sawed, scribed or laser-scribed into individual sticks, which are then cleaved or broken apart.

### Plating of the Sensor Sticks or Wafer

A stick of sensor elements that has been created, masked and fractured as just described is ready to be plated.

### Descum/RIE Etch

Before plating, a surface cleaning step may be performed of the titanium resistive layer. The surface cleaning may be performed by a cloud of oxygen plasma generated by a high frequency RF (13.65 MHz). The generated oxygen free radicals remove organic carbonaceous material and hydrogen by forming CO₂ and H₂O that is removed from the chamber. This decontamination step improves the nucleation on the surface resulting in a better sensor.

### Electroplating of Pd—Ag

A design of a group of sensors, such as a stick of five described here, may incorporate one or more test elements used during the plating process. This was initially used to find the approximate initial values for the plating of a functional sensor.

Test elements and their process reference points may be used to monitor the in situ conductivity changes. The test elements may be located at two places on each stick and can be monitored before and after the plating process.

One embodiment of the sensor works on a principle of shorting of the resistive region between two adjacent palladium particles. In order to grow the proper size particles, the resistance of the test elements may be measured periodically during the plating process. No resistance change of the test element may be expected until palladium particles grow to the size that inter-particle shorts start to occur.

The point at which resistance change is noted (time versus current) serves as a marker, and plating time for subsequent runs may be adjusted to end so many seconds before or after that marker.

The test elements may help determine the sensitivity of the sensor as a function of the substrate resistance change. The closure of more nanogaps between the nanoparticles leads to the effective decrease in resistance. The test elements may help check the severity of the “edge effects,” the excessive buildup of palladium along sensor edges when the 20 μm mask border is not used.

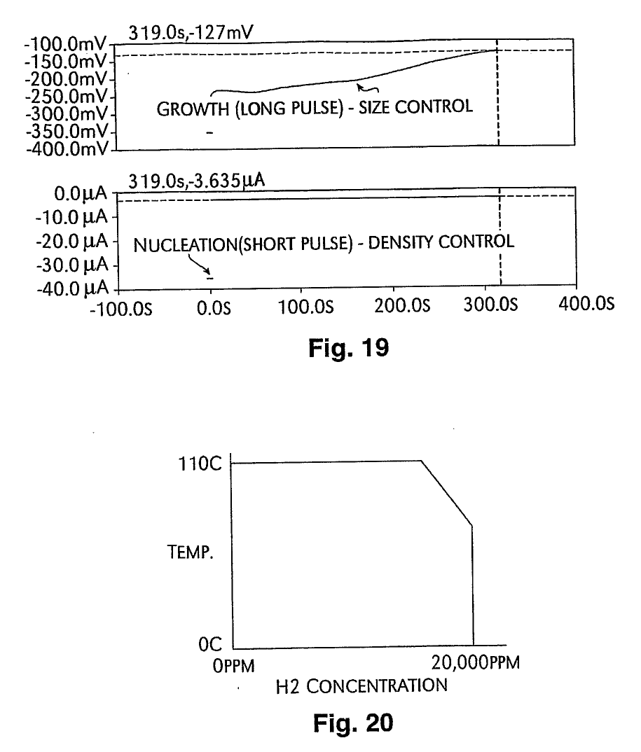

### Nucleation and Growth Steps (FIG. 19)

The electroplating of the Pd—Ag alloy on a conductive substrate is a two-step process consisting of nucleation and growth phases. The conductivity of the base substrate increases from the point of time of the nucleation time (typically less than 10 seconds) and the end of the growth step (typically around 10 minutes). The increase in the conductivity is a function of increased metal deposited on the substrate that is reflected by the increase in the plating potential (Eₛₜₐᵣₜ=−350 mV and E_{end}=−127 mV). Density of the nanoparticles is controlled by the nucleation charge and the size of the particles is controlled by the growth charge.

### Use of Nanoparticles Versus Thin Film Characteristics

During process development tweaks, it may be useful to recognize the essential difference in resistance changes for the nanoparticle behavior versus thin film behavior. An increase in hydrogen exposure causes thin films to increase in resistance. A similar increase for this nanoparticle based sensor causes a decrease in resistance. By this means, severe over-plating can be readily identified. In this case, all nanoparticles have adjoined to each other to form a continuous film, which is undesired behavior for this sensor.

### Design for Reduction of Stress in the Nanoparticle Sensor

Several kinds of stress (fatigue) were previously discussed. These relate to deformation of the particle by stressing it beyond its physical limits of elasticity. The plating process directly controls the limitations of stress in the target sensor.

The “tweak knobs” for stress reduction are:
1. Control of nucleation and growth times to preclude crowding of the particles under conditions of high temperature and high hydrogen concentration.
2. Trade off growth density versus desired low-end sensitivity. Increasing the density brings particles closer together, to almost or just touching. This is the point of maximum low-end sensitivity, and permits measurements to a few ppm or less. In this mode there is a limitation on the high concentration end of the dynamic range of the sensor.
3. High-density packing of nanoparticles for highest sensitivity may be permitted if the proper Pd:Ag alloy ratio is used. Higher ratios of silver, e.g., 60:40 Pd:Ag versus 90:10 Pd:Ag improves the elasticity of the particles. Under these conditions, the sensor holds its calibration much better under stress, giving a widely improved dynamic range. Response time is reduced accordingly.
4. High-concentration sensitivity, e.g., 1000 ppm to 40,000 ppm or more, is improved by nucleating the particles more sparsely.
5. Response time is improved by using less silver in the Pd:Ag alloy. As shown previously, a pure-Pd sensor has very fast response times.
6. For oil-based sensors, the diminishing of Ag concentrations in the ratio is to be avoided, unless rapid temperature drops in H₂-loaded sensors will not occur. This was also discussed previously.

The above are plating-time controls over the operation of the sensors of embodiments of this invention.

Conditioning of the sensor may accomplish two things. It may reduce the rate of sensor aging and drift by pre-oxidizing the surfaces. It also may pre-stress the sensors to a point beyond that which is encountered during in-spec operation, stabilizing its long-term operation.

Discussed previously, and especially for the oil-based sensors, there are conditions of hydrogen and temperature that induce morphology-based stress into the sensor, possibly altering its future operation. There are certain limits of combined hydrogen and temperature exposure which may stress the sensor beyond its capability to recover. The causes and means of this stress were discussed.

FIG. 20 depicts the limitation of hydrogen and temperature exposure that might be safe for a given sensor. The conditioning must exceed the safe allowable limits slightly, to be effective at the limit.

Actual limit values vary with the plating density and particle size, and Pd—Ag ratio. Once these parameters are fixed, the limits are also fixed, within process variances.

### Oxygen Conditioning Phase

The oxidation phase of conditioning elevates the sensor temperature “en vitro” in the presence of oxygen, causing the titanium surfaces to oxidize. The longer it is left in this condition, the more oxide is formed, and the less future changes of resistance occur.

For air-based sensors, this is done in normal air at perhaps 80-100° C. for several hours. For oil-based devices, this is done in oil that has been air-bubbled to encourage the dissolving of oxygen into the oil. Again, it is left for 4-8 hours at perhaps 100-110° C.

Generally, the temperature it is exposed to exceeds the maximum operational temperature by at least 10-20%.

### Hydrogen Conditioning Phase

FIG. 20 again shows the safe operating limits of safe simultaneous exposure to temperature and hydrogen. Actual limits are discovered empirically from a given set of plating parameters. Once plating parameters are fixed, these limits can be established by the destructive testing of sensors, to exceed these limits.

Conditioning takes place “en vitro” again, but (for oil) in oil that has been pre-bubbled with hydrogen of the required concentration. Sensors to be conditioned may be placed in a chamber such as a large syringe along with that oil. A syringe may be used to maintain a constant level of hydrogen, permitting none to escape as the temperature is increased. The plunger may extend as the oil itself expands, but all hydrogen may remain within the oil.

At the conclusion of this conditioning phase, the temperature may be brought back to room conditions very slowly, over a period of hours. Failure to do this may result instead in the formation of palladium hydride for reasons discussed earlier, permanently de-sensitizing the sensor, and possibly rendering it unstable.

The above limitation may not apply to sensors conditioned in a heated gaseous (air) environment in hydrogen.

### Calibration and Test of a Sensor

The calibration and test may procedure accomplish three things:
1. Exposure of sensor to high dosages of hydrogen.
2. Characterization of resistance changes due to hydrogen.
3. Characterization of resistance changes due to temperature.

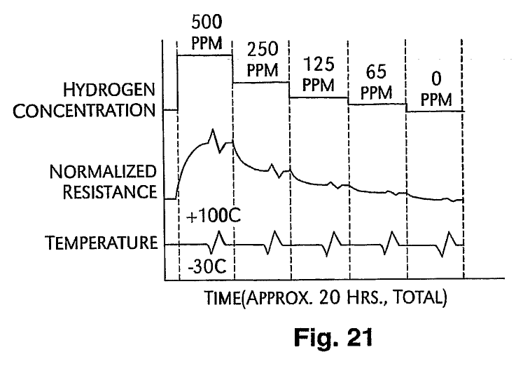

The procedure for making the calibration measurements is depicted graphically in FIG. 21. That figure illustrates separate calibration cycles for each level of H₂ concentration. Cycles are delineated by the dashed vertical lines in the figure. (The “Normalized Resistance” curve is shown inverted from actual changes. Resistance drops with increasing temperature and with increasing H₂ concentration.)

The same effective measurements may be made, whether it is done for sensors in oil or in air. The only fundamental difference in dynamics of the system is that for oil-based sensors, any slew from some elevated temperature to a lower one is done slowly. An example slew rate limitation is −40° C./hour.

Described earlier, the Pd alloy nanoparticle sensor is a nanoscale system with complicated physical processes taking place during interaction of nanoparticles with hydrogen and each other at different external conditions. Reliable operation of the sensor may require methodical characterization of the device in a wide range of temperatures, hydrogen concentrations, gas or liquid mixtures, and other parameters.

Although the present invention and its advantages have been described in detail, it should be understood that various changes, substitutions and alterations can be made herein without departing from the spirit and scope of the invention as defined by the appended claims.

What is claimed is:

1. A device for sensing hydrogen based on palladium or palladium alloy nanoparticles, wherein the nanoparticles are deposited on a resistive substrate, to permit sensing of less than 1% hydrogen; wherein the nanoparticles are deposited as islands on a continuous resistive layer.

2. The device as recited in claim 1, wherein the nanoparticles grow in size and alter a resistance of the sensor on exposure to hydrogen in air or dissolved hydrogen in oil.

3. The device as recited in claim 1, wherein the device senses the hydrogen in air.

4. The device as recited in claim 1, wherein the nanoparticles expand in the presence of hydrogen at a temperature which is higher than a palladium phase transition temperature at a given hydrogen concentration.

5. The device as recited in claim 1, wherein the device senses the hydrogen in oil.

6. The device as recited in claim 1, wherein the nanoparticles comprise palladium alloys to achieve fast response time at a temperature operation which is higher than a palladium phase transition temperature at a given hydrogen concentration.

7. The device as recited in claim 1, wherein the nanoparticles are deposited in spaced apart stripes.
